(19) United States
(12) Patent Application Publication
(10) Pub. No.: US 2010/0261737 A1
(43) Pub. Date: Oct. 14, 2010

(54) Method of Treating Erectile Dysfunction

(75) Inventors: Garrett Pilcher, Windsor, CA (US); Mark J. Dolan, Santa Rosa, CA (US); Robert Melder, Santa Rosa, CA (US); Christopher Bingham, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/421,768

(22) Filed: Apr. 10, 2009

(51) Int. Cl. A61K 31/519

(52) U.S. Cl. 514/262.1

(57) ABSTRACT

A combined therapy for treatment of a patient having erectile dysfunction at least in part caused by vascular disease within a pelvic vessel is disclosed that includes placement of a stent within a stenosed region of the pelvic vessel in conjunction with the administration of a drug for treatment of erectile dysfunction.

### FIELD OF THE INVENTION

The present invention relates to a method of treating erectile dysfunction. More particularly, a method of treating a patient having erectile dysfunction caused or aggravated by atherosclerosis in a body vessel that includes performing a revascularization procedure within the stenosed region of the affected vessel in conjunction with administering a drug for treatment of erectile dysfunction.

### BACKGROUND OF THE INVENTION

The National Institutes of Health estimates that 20 to 30 million American men suffer from mild, moderate or complete erectile dysfunction. Erectile dysfunction is the chronic, i.e., greater than three months duration, inability to maintain a penile erection for satisfactory sexual intercourse.

Vascular disease is a leading cause of erectile dysfunction. Atherosclerosis of the pelvic arteries alone may account for about 40% of erectile dysfunction sufferers over 40 years of age with erectile dysfunction. It has been shown that causes of erectile dysfunction include arterial disease anywhere along the arterial tree from the abdominal aorta to the helicine arteries. These arteries include, from proximal to distal, the lower abdominal aorta, common iliac, internal iliac (hypogastric), internal pudendal, and penile arteries, i.e., dorsal, deep, and helicine arteries. Other possible vascular-related causes of erectile dysfunction include, but are not limited to, diabetes, hypertension, high cholesterol, renal disease, trauma, surgery and smoking.

Erection of the penis is a parasympathetic nervous system process that ultimately results in the release of neurotransmitters, nitric oxide (NO), and vasodilation of the helicine arteries in the corpora cavernosa of the penis. More particularly, NO is first derived from nonadrenergic, non-cholinergic neurotransmission through a NO synthase catalyzed reaction involving endogenous L-arginine. However, a second and principle source of NO, allowing for sustained penile erection, is derived from the endothelium. The NO activates soluble guanylyl cyclase, which raises the intracellular concentration of cyclic guanosine monphosphate (cGMP). As the levels of cGMP increase, the relative levels of calcium shift and cause the cavernosal smooth muscle cells to relax. This relaxation causes dilation and enlargement of the corpora cavernosa, helicine arteries and luminar spaces. This vasodilation and enlargement exerts force on the subtunical albuginea thereby depressing outflow of blood through the venous system. This orchestrated process results in penile rigidity and erection. cGMP is hydrolized by phosphodiesterase type 5 (PDE-5). VIAGRA® (sildenafil citrate, produced by Pfizer, Inc., New York, N.Y.), as well as other PDE-5 inhibitors, work by inhibiting PDE-5, thus increasing the concentration and duration of cGMP in the smooth muscle cells and extending the duration of smooth muscle cell relaxation and penile erection.

While VIAGRA® and other PDE-5 inhibitors, taken orally, are effective in a many erectile dysfunction sufferers to produce an adequate erection for sexual intercourse, their effectiveness is dependent upon the severity of erectile dysfunction and/or any underlying disease. It has been documented that 30-60% of erectile dysfunction sufferers have sub-optimal responses to PDE-5 inhibitors. In cases where atherosclerotic disease restricts or diminishes blood flow beyond a stenosis in a pelvic or penile artery that may be insufficient to achieve an erection, the endothelium may also become less functional or even nonfunctional due to the decrease in blood flow such that the patient's normal ability to synthesize NO becomes impaired. Without the full compliment of functional endothelium in the pelvic vasculature, the system may be unable to create sufficient NO in order to drive the erection process. With impaired NO production possibly a result of atherosclerosis of a pelvic or penile artery, PDE-5 inhibitors may be ineffective or achieve a suboptimal response.

Accordingly, what is needed is a method of treating erectile dysfunction that may at least in part be caused by a lack of blood flow in the pelvic or penile arteries due to atherosclerosis, which may result in insufficient NO production by the endothelium, that is not effectively or optimally treated by administering PDE-5 inhibitors alone.

### BRIEF SUMMARY OF THE INVENTION

Embodiments hereof are directed to methods of treating erectile dysfunction caused at least in part by atherosclerosis in a pelvic artery. The method includes tracking a delivery catheter to a stenosis within the pelvic artery and delivering a vessel wall support structure, such as a stent, within the stenosis to restore blood flow through the pelvic artery to the penile arteries and subsequently administering a pharmaceutically effective amount of a drug for treatment of erectile dysfunction. In an embodiment, the drug for treatment of erectile dysfunction is a PDE5 inhibitor.

In various embodiments hereof, the pelvic artery may be one of the common penile artery, internal pudendal artery, internal iliac artery (hypogastric) and common iliac artery. In addition to stenting, various treatments for revascularization of a pelvic artery in accordance with embodiments hereof include breaking-up and/or removing the stenosis by rotoblading, brachytherapy, atherectomy, ultrasonic disintegration, clot retrieval, drug/biologic delivery, such as by a drug-coated balloon, vessel paving, and balloon angioplasty.

### BRIEF DESCRIPTION OF DRAWINGS

The foregoing and other features and advantages of the invention will be apparent from the following description of embodiments thereof as illustrated in the accompanying drawing. The accompanying drawing, which is incorporated herein and forms a part of the specification, further serves to explain the principles of the invention and to enable a person skilled in the pertinent art to make and use the invention. The drawing is not to scale.

FIG. 1 is an illustration of a guide catheter and stent delivery system for use in a method of treating erectile dysfunction according to an embodiment of the present invention.

### DETAILED DESCRIPTION OF EMBODIMENTS OF THE INVENTION

The following detailed description is merely exemplary in nature and is not intended to limit the invention or the application and uses of the invention. Furthermore, there is no intention to be bound by any expressed or implied theory presented in the preceding technical field, background, brief summary or the following detailed description.

As discussed above, erectile dysfunction is a disease that has several etiologies, one of which is insufficient blood flow to the penile arteries. Essentially, penile erection occurs when the two corpora cavernosa fill with blood and maintain pressure adequate for penetration. Each corpus cavernosum is fed by a deep artery of the penis located in the center of each cavernosum. The deep artery is a branch of the internal pudendal artery that is a branch of, and in some cases, a direct continuation of the internal iliac artery, also called the hypogastric artery, which is a terminal branch of the common iliac arteries. The internal pudendal artery feeds most of the external genitalia and perineal structures, wherein the perineum refers to the diamond-shaped area between the thighs from the pubic bone to the tip of the coccyx.

Each deep artery has many smaller coil-shaped arteries, called helicine arteries extending downstream therefrom that open directly into the corpora cavernosa. As discussed above, erection of the penis is a parasympathetic nervous system process that ultimately results in the release of neurotransmitters and nitric oxide (NO), which results in vasodilation of the helicine arteries in the corpora cavernosa. In addition to dilatation, the helicine arteries also straighten out from their normally coiled configuration to further increase blood flow into the cavernosa. Blood then fills these erectile compartments, and in the process compresses the penile veins that drain these tissues. Activation of the sympathetic nervous system returns the penis to a flaccid state.

Vascular disease occurring anywhere along the arterial path from the abdominal aorta through the internal pudendal artery can adversely affect blood flow to the penile arteries, which in turn may decrease NO production due to the effect of the decreased blood flow on the endothelium. As discussed in U.S. Pat. Appl. Pub. No. 2007/0283969 to Yamasaki et al., which is incorporated by reference herein in its entirety, stenting or the performance of another revascularization procedure within the pelvic arterial region may be effective in treating erectile dysfunction caused by vascular disease, if the patient otherwise exhibits normal blood flow within the penis, which may be diagnosed as disclosed in Yamasaki et al. One of the anticipated values of increasing blood flow through revascularization of a pelvic artery is improving the peak velocity of the inflow of blood into the pelvic arteries and tissues. The rapid inflow will cause the corpus cavernosum to expand rapidly against tunica thus occluding the venous system. This fluid dynamic condition will result in a net increase of blood residing in the penis thus an erection.

However, in some patients the restored blood flow provided by stenting or the performance of another revascularization procedure may not entirely address erectile dysfunction. In accordance with an embodiment hereof, such a patient may benefit from use of a PDE5 inhibitor, such as VIAGRA, after the revascularization procedure, even if that patient had previously experienced a suboptimal response to administration of the same PDE5 inhibitor prior to the revascularization procedure. Thus in accordance with an embodiment hereof, a method of treating erectile dysfunction that is caused by vascular disease occurring anywhere along the arterial path from the abdominal aorta through the internal pudendal artery includes the step of performing a revascularization procedure at a stenosis in one of the pelvic arteries and thereafter the step of administering a pharmaceutically effective amount of a PDE5 inhibitor for use when sexual intercourse is desired. The increased blood flow achievable by the revascularization procedure may help to restore proper function to compromised endothelium downstream of the treatment site, i.e., endothelium that due to insufficient blood flow may have been producing negligible or inadequate NO for driving the erection process, such that a subsequently administered PDE5 inhibitor will achieve a favorable or even an optimal response, and/or an improved response not previously realized by a patient on the PDE5 inhibitor alone. The patient's improved response to the PDE5 inhibitor after the revascularization procedure may include a quicker response time and/or sensitivity to the drug and/or increased tumescence. With blood flow restored and able to flow into the penis faster, a benefit of improved smooth muscle cell relaxation in the corpus cavernosum may be realized, as well as an improved compression of venous system thus less leaks that would inhibit tumescence.

Other drugs for treatment of erectile dysfunction that may be administered in various embodiments hereof include but are not limited to papaverine, phentolamine, prostaglandin A1, gene therapy, and may be as described in or developed from one or more of the following patents, U.S. Pat. No. 6,100,270 to Campbell, U.S. Pat. No. 6,300,335 to Campbell et al., and U.S. Pat. No. 6,469,012 to Ellis et al.

In various embodiments, the pelvic artery in which the revascularization procedure is performed may be one of the common iliac artery, external iliac artery, internal iliac (aka hypogastric artery) and/or the internal pudendal artery, common penile artery. In addition to stenting, various treatments for revascularization of a pelvic artery by removal, destruction or radially displacement of the stenosis in accordance with embodiments hereof include rotoblading, brachytherapy, atherectomy, ultrasonic disintegration, clot retrieval, drug/biologic delivery, such as by a drug-coated balloon, vessel paving, and balloon angioplasty.

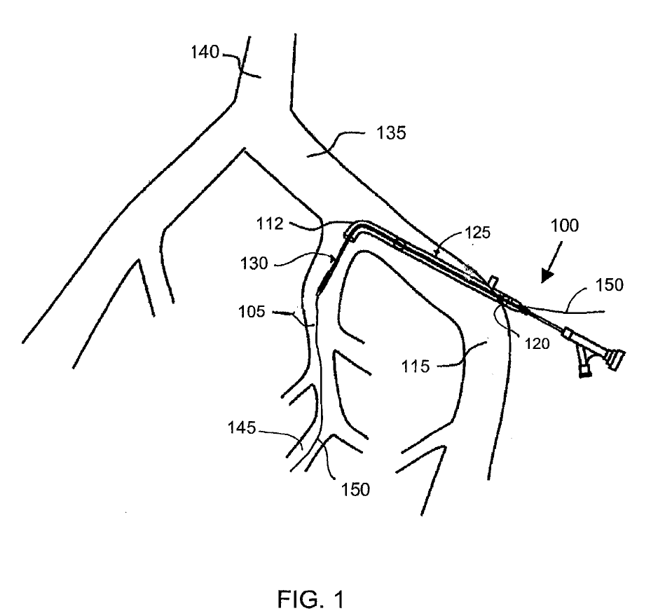

As shown in an exemplary embodiment of FIG. 1, a guide catheter and stent delivery system 100 may be used to more easily navigate the pelvic vasculature in the performance of a stenting procedure therein. System 100 is more fully disclosed in U.S. Patent Appl. Pub. No. US2006/0079951 A1 to Dolan et al., which is incorporated by reference herein in its entirety. System 100 is introduced into femoral artery 115 through a percutaneous access site 120 to be tracked over a guidewire 150, which in FIG. 1, extends within the pelvic vasculature to the treatment site (not shown) within the internal pudendal artery 145. System 100 includes a guide catheter portion 125 shown passing within femoral artery 115 and having an angled tip 112 directed into an ostium of internal iliac artery 105. A stent delivery catheter portion 130 of system 100 is shown extending into internal iliac artery 105 from guide catheter portion 125. Stent delivery catheter portion 130 is then maneuverable through internal iliac artery 105 into internal pudendal artery 145 to the site of the stenosis (not shown). For reference, the right common iliac artery and abdominal aorta are indicated at 135 and 140, respectively. For an application as shown in FIG. 1, the length of guide catheter portion 125 of system 100 may be less than 50 centimeters. In order to access distal treatment sites of the internal pudendal artery, stent delivery catheter portion 130 may be positionable within a vessel diameter of as little as 2 mm in accordance with embodiments of the present invention. In alternative embodiments, a delivery catheter for delivering a paving material may be used instead of stent delivery catheter portion 130 and such a delivery catheter would also be operable within a vessel of as little as 2 mm.

A stent for use in embodiments of the present invention may be balloon expandable or self-expanding and may be made from any suitable medically implantable material, such as, but not limited to, stainless steel, nitinol, tantalum, ceramic, nickel, titanium, aluminum, polymeric materials, cobalt alloys, platinum iridium, magnesium alloys, titanium ASTM F63-83 Grade 1, niobium, high carat gold K 19-22, and combinations thereof. In embodiments hereof, the stent may be a bare metal stent or a drug-eluting stent. Stents and structures for stents suitable for use in embodiments of the present invention are disclosed in U.S. Pat. No. 4,733,665 to Palmaz, U.S. Pat. No. 4,800,882 to Gianturco, U.S. Pat. No. 4,886,062 to Wiktor, U.S. Pat. No. 5,133,732 to Wiktor, U.S. Pat. No. 5,292,331 to Boneau, U.S. Pat. No. 5,421,955 to Lau, U.S. Pat. No. 5,776,161 to Globerman, U.S. Pat. No. 5,935,162 to Dang, U.S. Pat. No. 6,090,127 to Globerman, U.S. Pat. No. 6,113,627 to Jang, U.S. Pat. No. 6,663,661 to Boneau, and U.S. Pat. No. 6,730,116 to Wolinsky et al., each of which is incorporated by reference herein in its entirety. As well, any suitable guide catheter and/or stent delivery catheter may be utilized in embodiments of the present invention, with or without a guidewire, as would be apparent to one of ordinary skill in the art.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A method of treating erectile dysfunction caused at least in part by atherosclerosis in a pelvic artery, the method comprising the steps of:
tracking a delivery catheter to a stenosis within the pelvic artery;
delivering a wall support device carried by the delivery catheter to the stenosis to restore blood flow through the pelvic artery; and
administering a pharmaceutically effective amount of a drug for treatment of erectile dysfunction.

2. The method of claim 1, wherein the pelvic artery is one of the internal pudendal artery, internal iliac artery and common iliac artery.

3. The method of claim 1, wherein the drug for treatment of erectile dysfunction is a PDE5 inhibitor.

4. The method of claim 1, wherein the wall support device is a balloon expandable stent.

5. The method of claim 1, wherein the wall support device is a self-expanding stent.

6. A method of treating erectile dysfunction caused at least in part by atherosclerosis in a pelvic artery, the method comprising the steps of:
tracking a stent delivery catheter to a stenosis within the pelvic artery;
delivering a stent to the stenosis to restore blood flow through the pelvic artery; and
administering a pharmaceutically effective amount of a drug for treatment of erectile dysfunction.

7. The method of claim 6, wherein the pelvic artery is one of the internal pudendal artery, internal iliac artery, and common iliac artery.

8. The method of claim 6, wherein the drug for treatment of erectile dysfunction is a PDE5 inhibitor.

9. The method of claim 6, wherein the stent is a balloon expandable stent.

10. The method of claim 6, wherein the stent is a self-expanding stent.

11. A method of treating erectile dysfunction caused at least in part by atherosclerosis in a pelvic artery, the method comprising the steps of:
performing a revascularization procedure at a stenosis within the pelvic artery to restore blood flow through the pelvic artery; and
administering a pharmaceutically effective amount of a drug for treatment of erectile dysfunction.

12. The method of claim 11, wherein the pelvic artery is one of the internal pudendal artery, internal iliac artery, and common iliac artery.

13. The method of claim 11, wherein the drug for treatment of erectile dysfunction is a PDE5 inhibitor.
